(19) United States
(12) Patent Application Publication
(10) Pub. No.: US 2010/0233772 A1
(43) Pub. Date: Sep. 16, 2010

(54) MODULAR SYSTEM FOR INTRODUCING A STREAM OF PROCESSED GRAIN INTO AN ETHANOL PRODUCTION FACILITY, AND ASSOCIATED METHODS

(75) Inventors: Brooke Ellen Achenbach, Clive, IA (US); Terrance OShea, Des Moines, IA (US)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: SYNGENTA PARTICIPATIONS AG, Research Triangle Park, NC (US)

(21) Appl. No.: 12/552,371

(22) Filed: Sep. 2, 2009

Related U.S. Application Data
(63) Continuation-in-part of application No. 12/404,702, filed on Mar. 16, 2009.

(51) Int. Cl. C12P 7/10; C12M 3/00

(52) U.S. Cl. 435/165; 435/289.1

(57) ABSTRACT

A modular system is provided to introduce a grain containing a commercial enzyme into an ethanol production facility. The modular system comprises a plurality of portable storage units configured to receive a raw grain material. Each storage unit is adapted for transportation between ethanol production facilities. A portable processing unit is in communication with each of the storage units for receiving the grain material therefrom. The processing unit is configured to mix a grain containing a commercial enzyme with a commodity grain so that the enzyme is in effective amounts to sufficiently carry out downstream enzymatic applications. The processing unit is further configured to meter the processed grain material at appropriate admix levels to an ethanol production facility. The processing unit is adapted for transportation between ethanol production facilities. Associated apparatuses and methods are also provided.

### FIELD OF THE INVENTION

This invention relates to modular systems generally, and more particularly to modular systems for storing, metering, and milling a grain material for delivery as a first grain stream into an ethanol production facility for combination with a second grain stream, and associated methods.

### BACKGROUND OF THE INVENTION

Commercial fermentation is a biological process in which organic material (e.g., corn, soybean, cellulosic material) is enzymatically converted through the actions of enzymes (e.g. alpha-amylase, cellulases, glucoamylase, hemicellulases) into compounds, such as fermentable sugars. These simple compounds may then be utilized or fermented by microorganisms to produce molecules of commercial importance such as biofuels, polymers, feed additives, chemicals and plastics. The amount and type of sample processing required (e.g. harvesting, storage, transportation, pretreatment, milling, and enzymatic degradation) prior to fermentation is dependent on the source organic material being utilized to produce fermentable sugars. The type and amount of enzyme or enzymes used to convert the source organic material into fermentable sugars is also dependent on the source molecule being broken down into molecules for fermentation. In addition, enzymes used to breakdown source organic material must be added according to each enzyme's ideal functional range such as appropriate temperature and pH relative to the particular enzyme(s) being used. In order to create the most efficient fermentation process as is ideal in commercial fermentations, the enzyme must be added at adequate concentrations commensurate to the batch size and amount of source organic material or substrate available for the enzyme to act on. The addition of too little enzyme may result in partial conversion of the substrate and subsequent lower yields of the favorable commercial compounds. Adding too much enzyme may not be cost efficient due to the high cost of commercial enzymes.

Fuel ethanol derived from corn is primarily produced in the U.S. using either a dry or wet milling process. Generally, corn grain is delivered to commercial ethanol plants via truck or train where the grain is transferred to storage bins which in some cases can supply the plant for 7-10 days. In dry milling, the corn grain may be transported by a conveyor from the storage bin to a hammer mill where the corn grain is ground into flour. The flour is then transferred to a tank where water is added to create a hot slurry followed by the addition of the enzyme alpha-amylase to promote starch degradation. Often the pH of the hot slurry will need to be adjusted to the alpha-amylase's optimal functional range (i.e. pH 5.8). The slurry may then be pumped through a pressurized jet cooker. After proceeding through a jet cooker, the mixture is cooled and more alpha-amylase may be added to promote further starch degradation. The pH and temperature is finally adjusted and second enzyme, glucoamylase is added to the mixture. This mixture referred to as “mash” is transferred into a fermentation tank where yeast is added and fermentations are allowed to ferment generally 50-60 hours. Fermentation is followed by distillation and dehydration finally producing 200 proof anhydrous ethanol. Dry milling is the favored method of commercial ethanol producers. There are other known variations of this method wherein the corn is first fractionated prior to fermentation and only the starch fractionates are converted to ethanol using the conventional large scale fermentation methods as discussed above. The wet milling process consists of separating the kernel components into germ, fiber, protein and starch by first steeping the corn in water or sometimes in a dilute mixture of sulfuric acid for 24-48 hours. Once steeped, the corn is separated into germ, fiber, protein and starch through various methods such as milling, pressing, centrifugation and extrusion (e.g., pushing material through various sized screens). Corn germ, fiber and protein can be sold and used in various commercial applications (i.e. food, animal feed, textiles, etc). Starch obtained through the wet milling process may be sold as corn starch, further processed to corn syrup or fermented to ethanol essentially as described in the dry milling fermentation process.

The demand for renewable fuels has grown considerably over the past decade and is projected to continue significant growth. The 2005 U.S. Energy Bill contains a set target of 36 billion gallons of renewable fuels by the year 2022 with a large portion (approximately 15 billion gallons) being produced from corn starch. Sugars generated from the degradation of plant biomass could provide plentiful, economically competitive feedstocks for fermentation into chemicals, plastics, feed additives and fuels. A potential hurdle in the conversion of plant parts into chemicals, plastics, feed additives and fuels may be the limitations of current practices in regards to end product yields, cost effectiveness and lack of infrastructure in current commercial plants to support new emerging technologies (e.g., transgenic crops, novel harvesting methods, new varieties of feedstocks used for biomass). Transgenic plants expressing enzymes that degrade organic material into fermentable sugars may play a significant role in overcoming the limitations of these industries and in providing a way to increase the production of renewable fuels. Transgenic plants expressing enzymes that may be used in these processes are described in the art (see for example patent publications: US 2003/0125534, US2004/0018607 and US 2003/0135885 all herein incorporated by reference). The use of seed from a transgenic corn plant in a corn ethanol dry mill wherein the seed expresses an alpha-amylase that would degrade starch into fermentable sugars is one example of an enzyme expressing transgenic plant that may benefit the production of corn ethanol. In corn ethanol production, current practices are generally performed as described in the dry and wet milling methods described above wherein commodity corn is milled and enzymes are added at specific time points of the process with careful monitoring of pH, temperature, viscosity and concentration of the enzyme added. A possible limitation of the use of transgenic corn seed expressing a alpha-amylase as described in the above example, may be that the transgenic corn seed needs to be mixed with commodity corn at a specific admix ratio in order to ensure that enough alpha-amylase is present to act on the total corn starch. Other factors that may be affected by the starting admix ratio of enzyme expressing seed to commodity corn might be downstream viscosity, starting pH and amount of mixing needed to disperse the enzyme. This same limitation may be realized as well in other plant biomass conversion methods wherein a plant or plant part expressing an enzyme of interest must be admixed with a commodity plant or plant part. For example, a feedstock expressing a c cellulase.

Thus, it would be desirable to provide a system and method for quickly and efficiently introducing a transgenic plant or plant part into an biofuel production facility for combination with a second stream of stock material, such that the combination may be admixed at appropriate levels in the production of a biofuel. Furthermore, it would be advantageous to provide such a system and method capable of use between biofuel production facilities for rapidly and efficiently carrying out plant trials to test new methods.

### SUMMARY OF THE INVENTION

The article “a” and “an” are used herein to refer to one or more than one (i.e., to at least one) of the grammatical object of the article. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

The above and other needs are met by the present invention which, according to one aspect, provides a modular metering system for providing a first stream of grain containing enzyme to be mixed at a specific admix ratio to a second stream of commodity grain wherein the mixture is to be further utilized in an ethanol production facility. Herein, the phrase “grain containing enzyme” may be any grain containing one or more enzymes of interest utilized in a commercial process. A “grain containing enzyme” may be a transgenic grain wherein the plant expresses the enzyme of interest or the grain may be treated by an enzyme containing formulation (e.g. sprayed on or coated). One example may be a transgenic corn grain that expresses or has been treated with an alpha-amylase or glucoamylase enzyme. Herein, the term “commodity grain” refers to a grain not containing a processing enzyme. Herein, the term “admix ratio” refers to the amount of grain containing enzyme mixed by total into a commodity grain. For example, a “10% admix” refers to a mixture containing 10% grain with enzyme into a total mixture of grain. For example, an admix containing 10 kg of transgenic corn expressing amylase and 90 kg of conventional corn would be considered a 10% admix. The appropriate admix ratio to be used in an ethanol production facility is dependent on the amount of enzyme per gram of grain present in the grain containing enzyme. The amount of enzyme per gram of grain may vary due to types of treatments, growing conditions, stability of enzyme, or other factors. Thus, in the current embodiment it is necessary to first know the enzyme concentration of the grain prior to setting the admix ratio in order to have adequate amounts of enzyme to perform processes in downstream applications of a ethanol production facility. For example, a grain containing a low amount of enzyme would therefore require a higher admix ratio relative to a grain containing a high amount of enzyme which would require a low admix level. An enzyme's concentration can be measured through the use of commercially available enzyme assay kits. Enzyme assays such as MEGAZYME'S Ceralpha assay (Cat# K-CERA) can be used to measure amylase enzyme activity. Alpha-amylase activity may be monitored as grain is delivered to the site directly from the truck or any time prior to entering the ethanol production facility. In one aspect the sample may be taken for assay following milling before entering the ethanol production facility. In another aspect the material may be sampled for assay after entering the ethanol production facility but prior to entering the fermentation tank. Once the grain is assayed the modular metering system can then be adjusted by weight of grain to increase or decrease admix levels to their desired ratio. The system comprises a plurality of portable storage units configured to receive a grain material. Each storage unit is adapted for transportation between fields or ethanol production facilities. A portable processing unit is in communication with each of the storage units for receiving the grain material therefrom. The processing unit is configured to transport and mill a first stream of grain containing enzyme into a second stream of commodity grain into an ethanol production facility. The processing unit is further configured to mix the first stream of grain containing enzyme into a second stream of commodity grain at appropriate admix levels to ensure downstream processes are adequately supplied with sufficient amounts of enzyme in an ethanol production facility. The processing unit is adapted for transportation between ethanol production facilities.

Another aspect provides a modular metering system for providing a first stream of grain containing enzyme to be mixed at a specific admix ratio to a second stream of commodity grain wherein the mixture is to be further utilized in an ethanol production facility. The system comprises a plurality of portable storage units configured to receive a raw grain material. Each storage unit is adapted for transportation between ethanol production facilities. A conveyor assembly is configured to operably engage each of the storage units so as to form a serial connection between the storage units. The conveyor assembly is configured to receive and transport the raw grain material from each storage unit. A portable processing unit is configured to be in communication with the conveyor assembly so as to receive the raw grain material from the storage units. The processing unit is configured to transport and mill the grain containing enzyme received from the storage units. The processing unit is further configured to meter admix ratios of the first stream of grain containing enzyme into a second stream of commodity grain wherein the mixture is to be further utilized in an ethanol production facility. The processing unit is adapted for transportation between ethanol production facilities.

In some aspects of the invention the portable storage unit may be capable of storing at maximum 100, 500, 1000, 2000, 3000, 4000, 5000, 6000, 7000 or equal to or greater than 8000 bushels of corn grain. In a preferred embodiment the portable storage unit may be capable of storing a maximum of 5000 bushels of corn seed or greater. Another aspect of the invention multiple portable storage units may be used to store a total capacity of 500, 1000, 2000, 3000, 4000, 5000, 6000, 7000, 8000, 9000, 10000, 15000, 20000 or equal to or greater than 21000 bushels of corn grain.

Still yet another aspect provides a modular metering system for providing a first stream of grain containing enzyme to be mixed at a specific admix ratio to a second stream of commodity grain wherein the mixture is to be further utilized in an ethanol production facility. Such a method comprises providing a plurality of portable storage units at an ethanol production facility, wherein each storage unit is configured to receive and store a raw grain material therein. The raw grain material disposed in the storage units is delivered to a conveyor assembly operably engaged with and serially connecting the storage units. The method further comprises transporting the raw grain material to a portable processing unit via the conveyor assembly, transporting and milling the raw grain material and metering admix ratios of the first stream of grain containing enzyme into a second stream of commodity grain wherein the mixture is to be further utilized in an ethanol production facility.

A further aspect provides a method of producing ethanol. Such a method comprises providing a portable processing unit at an ethanol production facility, transporting and milling a grain containing enzyme, admix ratios of the first stream of grain containing enzyme into a second stream of commodity grain wherein the mixture is to be further utilized in an ethanol production facility.

Another aspect provides a method of providing a mixed grain material to an ethanol production facility for use in ethanol production. Such a method comprises introducing a metered first stream of grain into an ethanol production facility from a source externally disposed with respect thereto, wherein the first stream of grain comprises a grain containing enzyme wherein the enzyme may be selected from the group consisting of but not limited to a-amylase, glucoamylase, glucose isomerase, glucanase, β-amylase, α-glucosidase, isoamylase, pullulanase, neo-pullulanase, iso-pullulanase, amylopullulanase, cellulase, exo-1,4-β-cellobiohydrolase, exo-1,3-β-D-glucanase, β-glucosidase, endoglucanase, L-arabinase, α-arabinosidase, galactanase, galactosidase, mannanase, mannosidase, xylanase, xylosidase, protease, glucanase, esterase, phytase, and lipase. Preferably, the processing enzyme is a starch-processing enzyme selected from the group consisting of α-amylase, glucoamylase, glucose isomerase, β-amylase, α-glucosidase, isoamylase, pullulanase, neo-pullulanase, iso-pullulanase, and amylopullulanase. More preferably, the enzyme is selected from α-amylase, glucoamylase, glucose isomerase, glucose isomerase, α-glucosidase, and pullulanase. The processing enzyme is further preferably hyperthermophilic unless the application is a raw starch or low temperature fermentation facility. In accordance with this aspect of the invention, the enzyme may be a non-starch degrading enzyme selected from the group consisting of protease, glucanase, xylanase, esterase, phytase, and lipase. Such enzymes may further be hyperthermophilic. The method further comprises introducing a second stream of grain into the ethanol production facility, wherein the second stream of grain comprising a commodity grain material, and combining the first and second streams of grain at specific admix ratios to form a mixed grain material for use in an ethanol production process.

In another embodiment it may be desired to make modifications to the system in order to process various other crop plants to be used in a commercial fermentation facility. As used herein, “crop plants” refers to any plant that is cultivated for the purpose of producing plant material sought after by man or animal for either oral consumption, or for utilization in an industrial, pharmaceutical, or commercial process. The invention may be applied to any variety of plants, including, but not limited to maize, wheat, rice, barley, soybean, cotton, sorghum, beans in general, rape/canola, alfalfa, flax, sunflower, safflower, millet, rye, sugarcane, sugar beet, cocoa, tea, tropical sugar beet, Brassica, cotton, coffee, sweet potato, flax, peanut, clover; vegetables such as lettuce, tomato, cucurbits, cassava, potato, carrot, radish, pea, lentils, cabbage, cauliflower, broccoli, Brussels sprouts, peppers, and pineapple; tree fruits such as citrus, apples, pears, peaches, apricots, walnuts, avocado, banana, and coconut; and flowers such as orchids, carnations and roses. Other plants useful in the practice of the invention include perennial grasses, such as switchgrass, prairie grasses, Indiangrass, Big bluestem grass, miscanthus and the like. It is recognized that mixtures of plants may be used.

In one embodiment the mobile metering system comprises portable storage units oriented in a horizontal position and said storage bins do not require the addition of a load bearing concrete slab for support In one aspect a vibrating screening device is used to separate foreign matter from the grain prior to milling. In another embodiment a seed scalper may be used to separate foreign matter from the grain prior to milling.

In one embodiment the configuration may be changed in that corn seed may be cleaned of large trash, stems, leaves, and associated chaff prior to entering the surge bin. Said seed may be cleaned by using a vibrating screening device, a scalper, debeaders, dybvig separator, air screen separators, gravity seperators, combinations therof or any other seed cleaning devices well known in the art. Herein a “cleaning device” refers to any method or device that may be used to remove unwanted material from plant material. For example, a cleaning device may remove large trash, stems, leaves, and associated chaff from corn grain prior to liquefaction in an ethanol production facility. A cleaning device may include but is not limited to the use of vibrating screening devices, scalpers, debeaders, dybvig separators, air screen separators, gravity separators or any combination thereof.

In another embodiment, it is understood that the second stream of plant material may be metered prior to processing (i.e. milling). This may be accomplished, for example, by analyzing a test sample of corn seed for enzyme activity from the second stream of plant material, metering whole corn seed grain followed by processing of the second stream of corn seed grain to be admixed into a first stream of processed or unprocessed commodity corn seed grain.

Embodiments of the present invention thus provide significant advantages as disclosed herein in further detail.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a schematic representation of a modular metering system used to introduce a first stream of grain containing enzyme to be mixed at a specific admix ratio to a second stream of commodity grain wherein the mixture is to be further utilized in an ethanol production facility, according to one embodiment of the present invention;

FIG. 2 is a schematic representation of a plurality of portable storage units for a modular system used to introduce a first stream of grain containing enzyme to be mixed at a specific admix ratio to a second stream of commodity grain into an ethanol production facility, according to one embodiment of the present invention;

FIG. 3 is a schematic representation of a portable processing unit for a modular metering system used to introduce a first stream of grain containing enzyme to be mixed at a specific admix ratio to a second stream of commodity grain into a ethanol production facility, according to one embodiment of the present invention;

FIG. 4A is a side view of a transport assembly capable of transporting a storage unit used for receiving and storing a grain material, according to one embodiment of the present invention, wherein the storage unit is in a transport position with respect to the transport assembly; and

FIG. 4B is a side view of the transport assembly of FIG. 4A wherein the storage unit is provided in an erect position with respect to the transport assembly.

### DETAILED DESCRIPTION OF THE INVENTION

The present disclosure now will be described more fully hereinafter with reference to the accompanying drawings in which some but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Embodiments of the present invention are directed to systems and methods used to introduce a first stream of grain containing a protein to be mixed at a specific admix ratio to a second stream of commodity grain into an ethanol production facility. In one particular embodiment, corn grain enhanced with amylase may be received, transported and milled by a modular system transported to and erected at an ethanol production facility, wherein the grain containing enzyme may be metered for appropriate admix ratio to be added into the ethanol production facility for combination with stock or commodity corn, such that the combination thereof may be used in an ethanol production process.

As previously described, in order to increase efficiency and reduce costs of ethanol production, only a admix of the starting material (e.g., grain and other organic materials) used to produce ethanol need include an grain containing enzyme, for example corn grain enhanced with amylase. In such instances, the grain containing enzyme material may be processed, for example, by a milling process such that the grain containing enzyme is prepared for addition to a stock or commodity portion of the starting material. As such, systems and methods of embodiments of the present disclosure may be provided for delivering, metering, or otherwise introducing in a rapid and efficient manner a grain containing enzyme (for combination with a stock or commodity starting material) into an ethanol production facility such that the combination may be used in an ethanol production process carried out at the ethanol production facility. In this manner, processing of the grain containing enzyme may be completed outside and remote from the ethanol production facility such that the ethanol production facility need not be equipped with such capabilities. As such, capital expenditures of the ethanol production facility may be reduced. It is understood that the following system may be modified to handle various feedstocks for example soy bean, whole cob and kernel, sugarcane and etc.

In addition, embodiments of the present disclosure may also provide mobile, modular, or otherwise portable systems capable of being transported between ethanol production facilities or field sites. In this regard, such portable systems may be easily moved between ethanol production facilities for facilitating ethanol plant trials of various enzyme enhanced materials. For example, a producer of transgenic corn grain enhanced with amylase may deliver its product to various ethanol plant facilities for trials of the transgenic corn grain in ethanol production. In this manner, embodiments of the present invention may permit the producer to rapidly erect the portable system at a trial site for testing of a plant material, and then deconstruct the portable system after the trials are complete such that the portable system can be moved to a new trial site. As such, embodiments of the present disclosure may be configured for rapidly constructing a temporary system at a trial site, wherein the system is capable of storing, milling, and metering, for example, an enzyme enhanced material. Thus, plant trials may be completed in a timely and cost-efficient manner such that the producer of the transgenic material can move easily between ethanol production facilities. In other instances, the mobile system may be provided at the ethanol production facility in a more permanent manner so as to provide a low-cost solution as an alternative to such a system being provided as permanent fixture of the ethanol production facility itself.

In one aspect the modular metering system quickly establishes a method for providing a second stream of plant material into a production facility wherein the second stream of plant material contains a protein to be admixed with a first stream of plant material. The term “plant material” refers to any part of a plant such as seed, leaves, stem, roots, reproductive structures, or any combination thereof. A production facility may be a Ethanol dry mill, wet mill, sugarcane plant, cellulosic conversion plant, paper mill, or any facility involved in the processing of plant material into commercial products such as fuels, plastics, feed additives, specialty sugars, or commercial chemicals.

In another aspect the modular metering system quickly establishes a method for providing a second stream of plant grain into a production facility wherein the second stream of plant grain contains a protein to be admixed with a first stream of plant grain. In a preferred embodiment the grain is that of maize or sorghum.

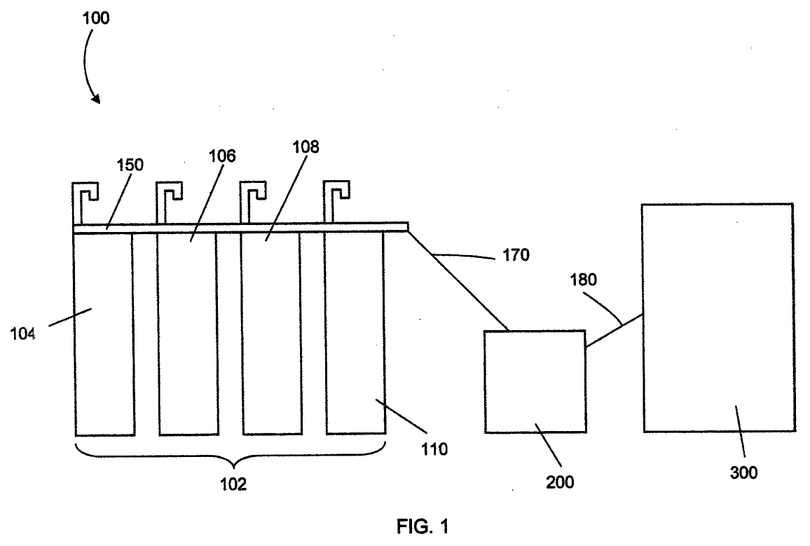

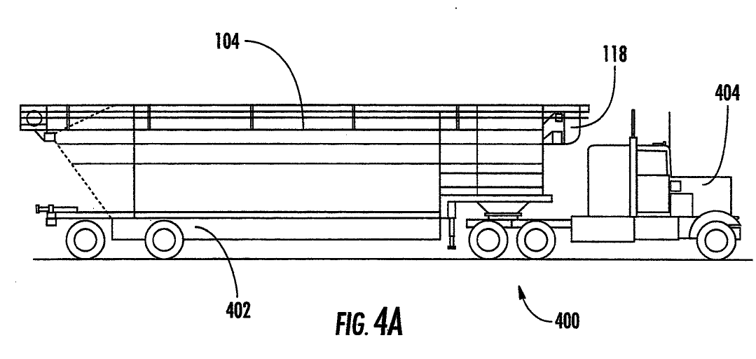

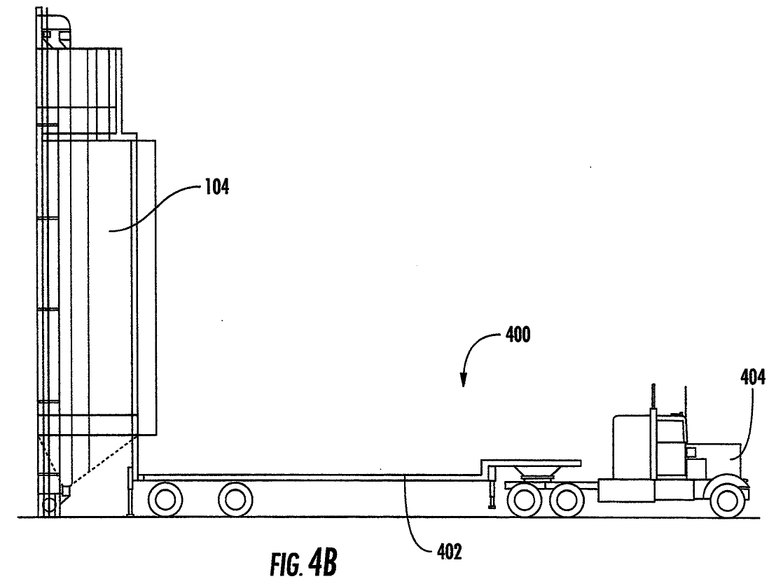

As shown in FIG. 1, a modular system 100 may be provided for storing, milling, and metering a grain material to an ethanol production facility 300. In one particular embodiment, the modular system 100 may store, mill, and meter transgenic corn material enhanced with amylase. In this manner, the transgenic corn material enhanced with amylase may be received by an ethanol production facility for combination with a stock or commodity corn grain used for ethanol production. In any instance, the modular system 100 may be capable of rapid transportation between ethanol production facilities 300 such that plant trials may be conducted at the various facilities 300. In this regard, the modular system 100 may be transportable by, for example, transport trailers (e.g., tractor-trailers) or other suitable transportation apparatuses. That is, in some instances, the modular system 100 and/or components thereof may be capable of being loaded onto or within a transport assembly 400 as shown in either FIG. 4A or 4B, such as, for example, on a tractor-trailer arrangement having a flat-bed trailer, for transportation to the ethanol production facility 300 wherein the modular system 100 and/or components thereof may be transported in a transport position, as shown in either FIG. 4A or 4B. For example, the transport assembly 400 may be configured to receive and transport a portable storage unit 102 to an ethanol production facility. In some instances, the transport assembly 400 may comprise a trailer portion 402 and a tractor portion 404, wherein the trailer portion 402 may be configured to receive, for example, the storage unit 102. In this regard, the modular system 100 may be comprised of multiple components such that the modular system 100 may be divided into individual components for transportation. In some instances, the components of the modular system 100 may be secured to or otherwise engaged with the transport assembly 400 (e.g., the trailer portion 402) during operation of the modular system 100. For example, the individual components may be hydraulically or otherwise engaged to the transport assembly 400/trailer portion 402 for cooperating therewith to provide stability or otherwise assist with positioning of the individual components to an erect position, as shown in FIG. 4B. In some aspects, during operation of the modular system 100, each component or otherwise a portion of the components of the modular system 100 may remain engaged with the transport assembly 400/trailer portion 402 on which the component is transported. In other instances, the individual components may be separated from the transport assembly 400 during operation of the modular system 100. In any instance, the modular system 100 may be configured for quick and rapid erection and deconstruction so as to efficiently facilitate movement between ethanol production facilities 300. Not included in FIG. 1, a 40×70×1 concrete pad may be poured to support each storage unit 102.

In one embodiment, the modular system 100 may comprise a plurality of portable storage units 102 for receiving and storing a raw grain material such as, for example, an enzyme enhanced grain material (e.g., transgenic corn grain enhanced with amylase). Of course, the raw grain material (e.g., enzyme containing grain) may be of any type suitable for ethanol production. While illustrating four portable storage units 102 (i.e., storage units 104, 106, 108, and 110), one of ordinary skill will recognize that any number of storage units 102 may be provided and that such illustrations are provided as an example and, thus, are not meant to limit the present disclosure. The storage units 102 may be in communication with each other such that the raw grain material stored therein may be transported from the storage units 102 by a single assembly such as, for example, a conveyor assembly 150. In this manner, the storage units 102 may be serially engaged by the conveyor assembly 150 for transporting the raw grain material to a portable processing unit 200 for processing (e.g., grain milling) thereof. In some instances, a first delivery arrangement 170 may be provided to transport the raw grain material from the conveyor assembly 150 to the processing unit 200. In other instances, the conveyor assembly 150 may directly transport the raw grain material to the portable processing unit 200 or, alternatively, the first delivery arrangement 170 may be directly engaged with one or more of the storage units 102 such that the raw grain material is directly moved from the storage units 102 to the processing unit 200. In any event, the raw grain material may be processed and passed through the processing unit 200 such that the resultant material is a processed grain material ready for combination with stock or commodity grain material to be used in ethanol production. The modular system 100 may further include a second delivery arrangement 180 for transporting, delivering, metering or otherwise introducing the processed grain material into the ethanol production facility 300. In any instance, as mentioned previously, each of the components (e.g., storage units 102, conveyor assembly 150, first delivery arrangement 170, second delivery arrangement 180, and/or processing unit 200) of the modular system 100 may be configured to be readily transportable between ethanol production facilities such as by, for example, the transport assembly 400.

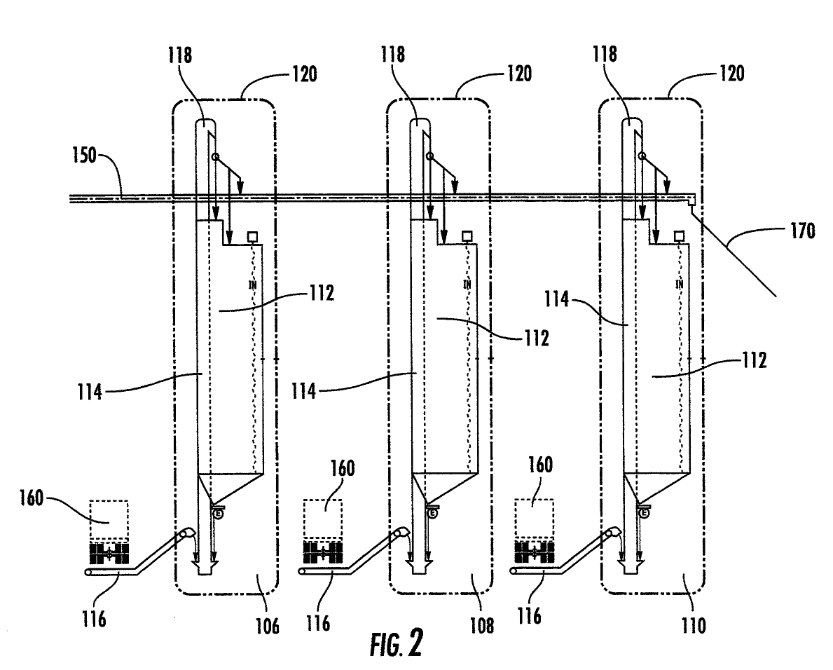

As shown in FIG. 2, each of the storage units 106, 108 and 110 (storage unit 104 not shown) may be configured as a silo structure 112 capable of storing the enzyme enhanced grain material therein. In some instances, the silo structure 112 may include an elevator system 114 or otherwise a vertical conveyor system (e.g., a vertical screw conveyor) for transporting the enzyme enhanced grain material to a first spout 118 such that the raw grain material can be delivered to the conveyor assembly 150. The first spout 118 may be arranged such that any overspill (i.e., grain material spilling over the conveyor assembly 150) of the raw grain material is returned to the silo structure 112 for interaction with the elevator system 114. In some instances, a receiving assembly 116 may be provided for receiving the raw grain material and delivering the raw grain material to the elevator system 114/silo structure 112. The receiving assembly 116 may be configured to receive the raw grain material from a grain delivery vehicle 160. For example, the receiving assembly 116 may comprise a conveyor capable of receiving the enzyme enhanced grain material from a grain delivery vehicle 160 regulated for transportation of such materials.

In some instances, each storage unit 104, 106, 108, and 110 may include a shell or housing 120 for encasing the silo structure 112. The housing 120 may be configured to improve ease of transportation. That is, the regularly shaped (e.g., as a substantially box-like structure) housing 120 may provide surfaces corresponding to the transport assembly 400, thereby improving ease of erection, disassembly, and/or transportation as compared to the irregularly shaped silo structure 112. As illustrated, the housing 120 may be a tower configuration having a substantially vertical orientation. However, in other instances, the housing 120 and/or storage units 104, 106, 108, 110 may have a substantially horizontal orientation. That is, the storage units 102 may transport the raw grain material horizontally therethrough, rather than vertically using, for example, the elevator system 114 associated with the silo structure 112. In such a horizontal orientation, the storage units 102 may employ horizontally extending conveyor assemblies (e.g., screw conveyors) for transporting the raw grain material disposed therein.

As previously mentioned herein, the storage units 102 may be connected by the conveyor assembly 150 such that the raw grain material may be transported from the storage units 102 to the processing unit 200. For example, the elevator system 150 (or a conveyor-type system) may transport the raw grain material stored in the silo structure 112 to the spout 118, wherein the raw grain material may be directed onto the conveyor assembly 150, for example, under the influence of gravity. In this manner, the raw grain material may be loaded onto the conveyor assembly 150 from storage unit 106 and passed by the storage units 108, 110 as the raw grain material is directed toward the processing unit 200. As such, the raw grain material may be received by the conveyor assembly 150 from all the storage units 102 simultaneously to maximize the amount of raw grain material directed toward the processing unit 200 or, alternatively, the raw grain material may be received from any one or combination of the storage units 102 in a controlled manner. For example, the storage unit 110 may be, in one instance, the only storage unit having the raw grain material stored therein. In this regard, the conveyor assembly 150 would only be transporting the raw grain material from the storage unit 110. Of course, one of ordinary skill in the art will recognize that any combination of storage units 102 may be operating at a given time to provide the raw grain material to the conveyor assembly 150, wherein, in other instances, only one of the storage units 102 may be operating, regardless of whether the raw grain material is stored in a non-operative storage unit 102 (i.e., a storage unit not feeding the raw grain material to the conveyor assembly 150). In some instances, the conveyor assembly 150 may comprise a screw conveyor, as known by those of ordinary skill in the art. The conveyor assembly 150 may be comprised of multiple components, wherein each component may be transported to the ethanol facility 300 with a respective one of the storage units 102. In other instances, the entire conveyor assembly 150 may be transported with one of the storage units 102, with the processing unit 200, or alone on a separate transport assembly, vehicle, or other transport mode.

In some instances, the first delivery arrangement 170 may be positioned to receive the raw grain material from the conveyor assembly 150. As such, the first delivery arrangement 170 may be configured to direct the raw grain material to the processing unit 200. In some instances, the first delivery arrangement 170 may be configured to telescopically adjust such that the first delivery arrangement 170 can extend or retract a suitable length for communicating with the conveyor assembly 150 and the processing unit 200. The first delivery arrangement 170 may be configured as an enclosed chute for directing the raw grain material to the processing unit 200. In such instances, the raw grain material may be transported to the processing unit 200 through the delivery chute under the influence of gravity or under pressure provided to the delivery chute so as to prevent blockage thereof as the raw grain material moves therethrough. The first delivery arrangement 170 may be transported with the storage units 102 and/or the processing unit 200, or alone on a separate transport trailer, vehicle, or other transport mode.

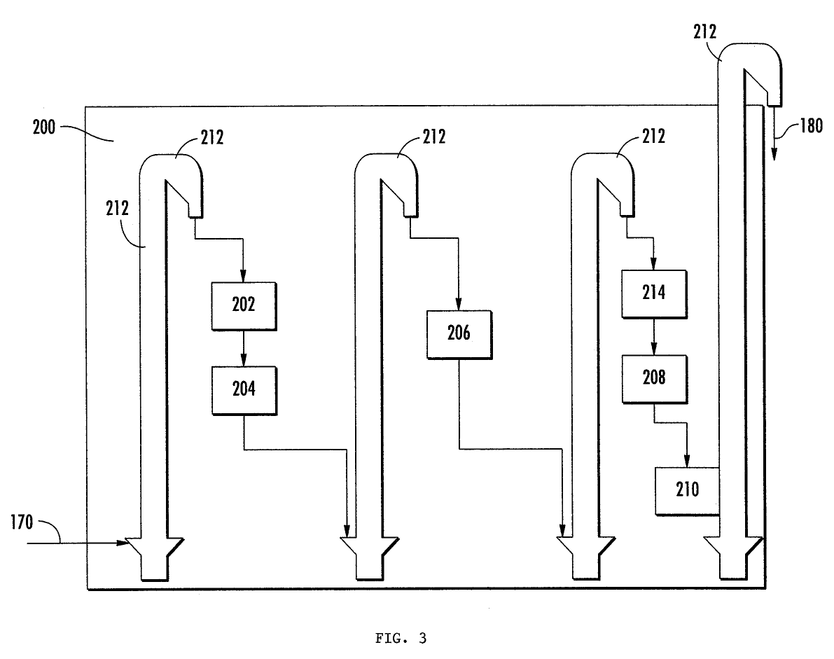

As shown in FIG. 3, the processing unit 200 may include various components for processing the raw grain material such that the raw grain material is prepared for use by the ethanol production facility 300 in an ethanol production process/procedure. In some instances, the various components of the processing unit 200 may be transported by a single transport assembly such as a trailer or vehicle (e.g., flat bed tractor-trailer) or otherwise by multiple transport trailers or vehicles. In some embodiments, the processing unit 200 may include, for example, a cleaning device 202, a reservoir container 204, a milling device 206, and a weighing device 208, or combinations thereof, wherein such components are provided to process and meter the raw/processed grain material. The cleaning device 202 may be in communication with the first delivery arrangement 170 for receiving the enzyme enhanced grain material therefrom, and thusclean the raw grain material of unwanted materials for example, large trash, stems, leaves, and associated chaff such that the raw grain material is suitably acceptable for milling and eventual ethanol production. The raw grain material may then be transported to the reservoir container 204. From the reservoir container, the raw grain material may be directed to the milling device 206 for milling into a processed grain form suitable for combination with the stock or commodity grain material provided by or otherwise at the ethanol production facility 300. In some instances, the milling device 206 may have a plenum device 207 in operable engagement therewith for assisting in the milling process. After milling, the processed grain material may be directed to the weighing device 208 (e.g., a weigh belt) such that metered portions thereof may be dispensed/delivered to the ethanol production facility 300. Other components for processing the raw grain material and/or metering the processed grain material may also be provided. For example, an in-line sampling device 214 may be provided in the processing line for retrieving a sample for off-line testing thereof, such as, for example, for determining enzyme activity, heterologous protein concentration or simple protein detection of the raw grain material being provided to the ethanol production facility 300.

According to some embodiments, grain transfer members 210, such as, for example, screw conveyors and tube screw conveyors, may be provided to connect the various components of the processing unit 200. Further, in some instances, a plurality of vertically-disposed chute members 212 may be provided in combination with the grain transfer members to transport the raw/processed grain material between the various components of the processing unit 200. A power source for operating the various components of the processing unit 200 may also be provided therewith or separately therefrom as a component that is remotely transportable with respect to the other components of the processing unit 200. In some instances, one of the chute members 212 may be provided after the weighing device such that the chute member 212 can transport the metered portion of the processed grain material to the second delivery arrangement 180, which is in communication with the ethanol production facility 300. For example, the second delivery arrangement 180 may be telescopically adjustable to allow extension and retraction thereof for adjusting to a grain entry point of the ethanol production facility 300. That is, the second delivery arrangement 180 may extend within the ethanol production facility such that the processed grain material can be directed, introduced, delivered, or otherwise provided into the ethanol production facility 300 for combination with the stock or commodity grain material.

Accordingly, embodiments of the present invention may thus serve to rapidly introduce a first grain stream into an ethanol production facility 300 for combination with a second grain stream, such that the combination of the first and second grain streams may be used for ethanol production. As previously disclosed, in one particular instance, the first grain stream may be a processed transgenic corn material enhanced with an enzyme and the second grain stream may be a stock or commodity corn material. In this manner, a metered portion of the first grain stream may be combined with the second grain stream in order to increase efficiency and reduce costs associated with ethanol production by reducing the total amount of the first grain stream needed for ethanol production. That is, while the overall material used for ethanol production could entirely comprise the transgenic corn material enhanced with an enzyme, an ethanol producer is able to reduce costs through combination of such enhanced materials with the stock or commodity grain material. As such, system embodiments of the present disclosure may provide a source, which is remote from the ethanol production facility and transportable thereto, capable of providing the first grain stream, wherein the system may be transportable between ethanol production facilities and configured to provide metered portions of processed grain material thereto. In this regard, plant trials using various transgenic corn breeds may be quickly and efficiently conducted at the ethanol production facility and other such facilities.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A modular system for providing a second stream of plant material into a production facility wherein the second stream of plant material contains a protein to be admixed with a first stream of plant material within the production facility, the modular system comprising:
one or more portable storage units configured to receive a second stream of plant material containing a protein to be admixed with the first stream of plant material, each storage unit being adapted for transportation between production facilities; and
a portable processing unit adapted for transportation between production facilities in communication with each of the storage units for receiving the second stream of plant material therefrom, the processing unit being configured to
a) receive the second stream of grain material from said one or more portable storage units;
b) deliver said second stream of grain material to a cleaning device then to a reservoir tank;
c) process and meter the cleaned second stream of grain material of b); and
d) deliver said second stream of grain material from c) into a first stream of plant material within the production facility.

2. A modular system for providing a second stream of grain material into a ethanol production facility wherein the second stream of grain contains a protein to be admixed with a first stream of grain within the ethanol production facility, the modular system comprising:
one or more portable storage units configured to receive a second stream of grain containing a protein to be admixed with the first stream of grain, each storage unit being adapted for transportation between ethanol production facilities;
a conveyor assembly configured to operably engage each of the storage units so as to from a serial connection therebetween, the conveyor assembly being configured to receive and transport the second stream of grain from each storage unit; and
a portable processing unit adapted for transportation between production facilities in communication with each of the storage units for receiving the second stream of grain material therefrom, the processing unit being configured to
a) receive the second stream of grain material from said one or more portable storage units;
b) deliver said second stream of grain material to a cleaning device then to a reservoir tank;
c) process and meter the cleaned second stream of grain material of b); and
d) deliver said second stream of grain material from c) into a first stream of plant material within the ethanol production facility.

3. A modular system according to claim 2, wherein each portable storage unit comprises a substantially vertical grain silo, the grain silo having an upper portion and a lower portion configured to receive the second stream of grain material, the grain silo having an elevator system configured to transport the raw grain material from the lower portion to the upper portion so as to be received by the conveyor assembly disposed thereabout.

4. A modular system according to claim 2, wherein each portable storage unit is oriented substantially horizontal.

5. A modular system according to claim 2, wherein the conveyor assembly comprises a screw conveyor assembly.

6. A modular system according to claim 2, further comprising a telescopic member configured to facilitate communication between the conveyor assembly and the processing unit, the telescopic member being configured to deliver the grain material from the conveyor assembly to the processing unit.

7. A modular system according to claim 2, wherein the processing unit comprises a milling device for milling the second stream of grain material.

8. A modular system according to claim 2, wherein the processing unit comprises a scalper or screening device.

9. A modular system according to claim 2, wherein the storage units and the processing unit are each configured to be transportable by a respective transport trailer.

10. A method of providing a second stream of grain material into a ethanol production facility wherein the second stream of grain contains a protein to be admixed with a first stream of grain within the ethanol production facility, the method comprising:
providing one or more portable storage units at an ethanol production facility, each storage unit being configured to receive a second stream of grain material therein;
transporting the second stream of grain material disposed in the storage units to a portable processing unit adapted for transportation between production facilities in communication with each of the storage units, the processing unit being configured to:
a) receive the second stream of grain material from said one or more portable storage units;
b) deliver said second stream of grain material to a cleaning device then to a reservoir tank;
c) process and meter the cleaned second stream of grain material of b); and
d) deliver said second stream of grain material from c) into a first stream of plant material within the ethanol production facility.

11. A method of producing ethanol, comprising:
providing one or more portable storage units at an ethanol production facility, each storage unit being configured to receive a second stream of grain material therein;
transporting the second stream of grain material disposed in the storage units to a portable processing unit adapted for transportation between production facilities in communication with each of the storage units, the processing unit being configured to:
a) receive the second stream of grain material from said one or more portable storage units;
b) deliver said second stream of grain material to a cleaning device then to a reservoir tank;
c) process and meter the cleaned second stream of grain material of b); and
d) deliver said second stream of grain material from c) into a first stream of plant material within the ethanol production facility.
